(19) United States
(12) Patent Application Publication
(10) Pub. No.: US 2024/0271609 A1
(43) Pub. Date: Aug. 15, 2024

(54) MAGNETICALLY DRIVEN PRESSURE GENERATOR

(71) Applicant: NOCIRA, LLC, Tempe, AZ (US)

(72) Inventors: David George, Scottsdale, AZ (US); Kevin E. Willey, Fort Collins, CO (US); John Claude, Redwood City, CA (US)

(21) Appl. No.: 18/514,788

(22) Filed: Nov. 20, 2023

Related U.S. Application Data
(63) Continuation of application No. 17/005,776, filed on Aug. 28, 2020, now Pat. No. 11,859,606.
Application No. 17/005,776 is a continuation of application No. 15/657,039, filed on Jul. 21, 2017, now Pat. No. 10,760,566.
(60) Provisional application No. 62/365,874, filed on Jul. 22, 2016.
(60) Provisional application No. 62/413,491, filed on Oct. 27, 2016.

(51) Int. Cl. F04B 43/04; A61F 11/00; A61F 11/30; A61H 23/02; F04B 45/027; F04B 49/06; F04B 49/24; F04B 51/00; F04B 53/06; G05B 15/02

(52) U.S. Cl. CPC F04B 43/04; A61F 11/00; A61H 23/0218; A61H 23/0236; F04B 45/027; F04B 49/065; F04B 49/24; G05B 15/02; A61F 11/30; F04B 51/00; F04B 53/06

(57) ABSTRACT

A magnetically driven pressure generator including a housing, flexible member, first magnetic force generator, and second magnetic force generator. The magnetically driven pressure generator oscillates the flexible member to increase or decrease the volume of a chamber, inversely increasing or decreasing the pressure of the chamber.

This United States Non-Provisional Patent Application is a continuation of U.S. patent application Ser. No. 17/005,776, filed Aug. 28, 2020, which is a continuation of U.S. patent application Ser. No. 15/657,039, filed Jul. 21, 2017, and issued as U.S. Pat. No. 10,760,566 on Sep. 1, 2020, which claims the benefit of U.S. Provisional Patent Application No. 62/413,491, filed Oct. 27, 2016, and U.S. Provisional Patent Application No. 62/365,874, filed Jul. 22, 2016, each hereby incorporated by reference herein.

### I. TECHNICAL FIELD

A magnetically driven pressure generator operable to adjust fluid pressure of an amount of fluid in an enclosed space to match pre-selected amplitude or frequency values or generate a continuous or intermittent fluid flow of the amount of fluid from the enclosed space to match pre-selected fluid pressure or fluid flow rate values over a period of time.

### II. SUMMARY OF THE INVENTION

A broad object of the invention can be to provide a magnetically driven pressure generator, including one or more of: a housing having an open end and a closed end, a flexible member having a peripheral margin sealably coupled to the open end to define an enclosed space, a first magnetic force generator disposed on the flexible member, and a second magnetic force generator disposed proximally adjacent to the flexible member, where either the first magnetic force generator or second magnetic force generator comprises an electromagnetic force generator, and a controller configured to continuously or intermittently control the magnitude and direction of a current flowing in the electromagnetic force generator to correspondingly continuously or intermittently control amplitude and frequency of flexure of the flexible member, to decrease or increase the volume of the enclosed space to correspondingly increase or decrease pressure within the enclosed space, or to correspondingly generate fluid flow in an amount of fluid.

Another broad object of the invention can be a method of making a magnetically driven pressure generator, including sealably engaging a peripheral margin of a flexible member to an open end of a housing, disposing a first magnetic force generator on the flexible member, disposing a second magnetic force generator proximate to the open end or the closed end of the housing, where either the first magnetic force generator or second magnetic force generator comprises an electromagnetic force generator, and providing a controller configured to continuously or intermittently control the magnitude and direction of a current flowing in the electromagnetic force generator to correspondingly intermittently or continuously control amplitude and frequency of flexure of the flexible member to correspondingly decrease or increase the volume of a closed space, to correspondingly increase or decrease pressure within the enclosed space, or to correspondingly generate fluid flow in an amount of fluid.

Another broad object of the invention can be a method of using a magnetically driven pressure generator, including obtaining a magnetically driven pressure generator, including a housing, a flexible member, a first magnetic force generator disposed on the flexible member, and a second magnetic force generator disposed proximally adjacent the flexible member, where either the first magnetic force generator or second magnetic force generator comprises an electromagnetic force generator operable to generate an amount of flexure of the flexible member to decrease or increase the volume of a closed space, pre-selecting the amplitude and frequency of a pressure change in the closed space by configuring a controller to correspondingly continuously or intermittently control the magnitude and direction of a current flowing in the electromagnetic force generator to correspondingly generate an amount of flexure of the flexible member to decrease or increase the volume of a closed space to continuously or intermittently generate the pre-selected amplitude and frequency in the closed space.

Naturally, further objects of the invention are disclosed throughout other areas of the specification, drawings, photographs, and claims.

### III. A BRIEF DESCRIPTION OF THE DRAWINGS

FIG. 1 is a perspective view of an embodiment of the magnetically driven pressure generator.

FIG. 2 is a front view of an embodiment of the magnetically driven pressure generator.

FIG. 3 is a top view of an embodiment of the magnetically driven pressure generator.

FIG. 4 is a side view of an embodiment of the magnetically driven pressure generator.

FIG. 5 is an exploded view of an embodiment of the magnetically driven pressure generator.

FIG. 6 is a perspective view of an embodiment of the flexible member.

FIG. 7 is a top view of an embodiment of a flexible member.

FIG. 8 is a front view of an embodiment of the flexible member.

FIG. 9 is a side view of an embodiment of the flexible member.

FIG. 10 is a front view of an embodiment of the flexible member including a magnet as a first magnetic force generator.

FIG. 11 is a cross section 11-11 of the flexible member shown in FIG. 10.

FIG. 12 is a front view of an embodiment of the flexible member including an electromagnet as a first magnetic force generator.

FIG. 13 is a cross section 13-13 of the flexible member shown in FIG. 12.

FIG. 14 is a perspective view of an embodiment of an external ear canal pressure regulation device including an embodiment of a magnetically driven pressure generator.

FIG. 15 is a top view of an embodiment of the external ear canal pressure regulation device including an embodiment of the magnetically driven pressure generator.

FIG. 16 is a bottom view of an embodiment of the external ear canal pressure regulation device including an embodiment of the magnetically driven pressure generator.

FIG. 17 is a first side view of an embodiment of the external ear canal pressure regulation device including an embodiment of the magnetically driven pressure generator.

FIG. 18 is a second side view of an embodiment of the external ear canal pressure regulation device including an embodiment of the magnetically driven pressure generator.

FIG. 19 is a front view of an embodiment of an embodiment of the external ear canal pressure regulation device including an embodiment of the magnetically driven pressure generator.

FIG. 20 is a back view of an embodiment of the external ear canal pressure regulation device including an embodiment of the magnetically driven pressure generator.

FIG. 21 is an exploded view of an embodiment of the external ear canal pressure regulation device including an embodiment of the magnetically driven pressure generator.

FIG. 22 is an exploded view of an embodiment of the external ear canal pressure regulation device including an embodiment of the magnetically driven pressure generator.

FIG. 23 is a first side view with the casing open to show the assembly of components of an embodiment of the external ear canal pressure regulation device including an embodiment of the magnetically driven pressure generator.

FIG. 24 is a schematic block diagram of a controller included in particular embodiments of the magnetically driven pressure generator or the external ear canal pressure regulation device.

FIG. 25 is an illustration of a method of using an embodiment of the external ear canal pressure regulation device shown in FIGS. 14 through 24 including an embodiment of the magnetically driven pressure generator shown in FIGS. 1 through 24.

### IV. DETAILED DESCRIPTION OF THE PREFERRED EMBODIMENTS

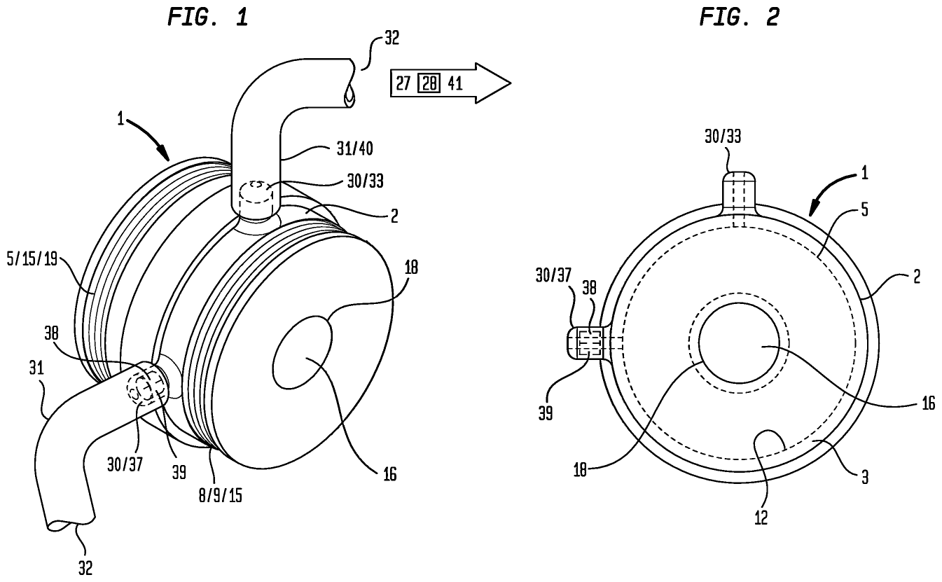

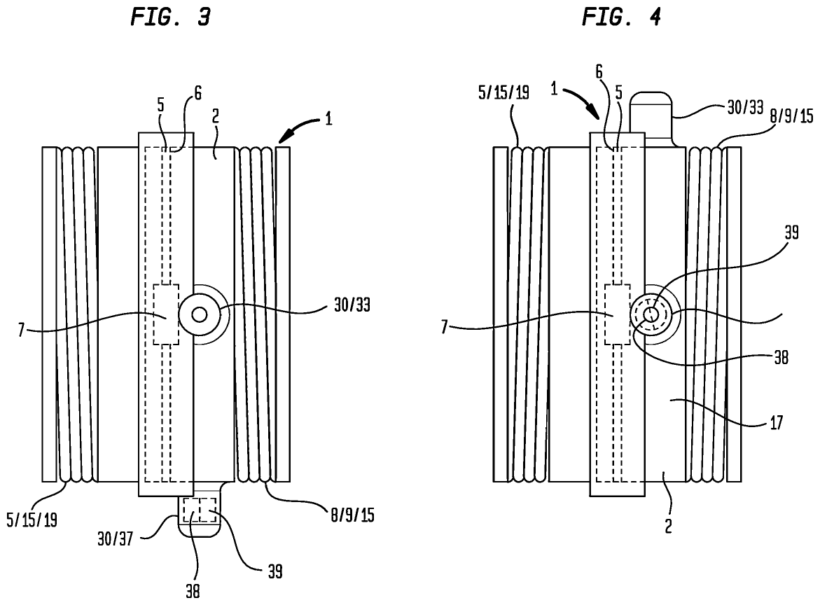

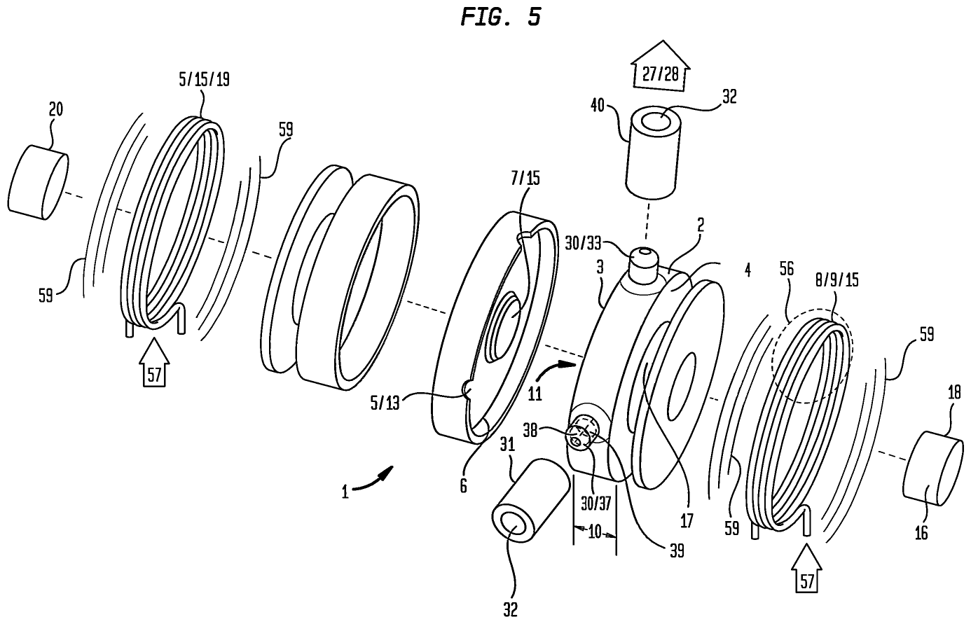

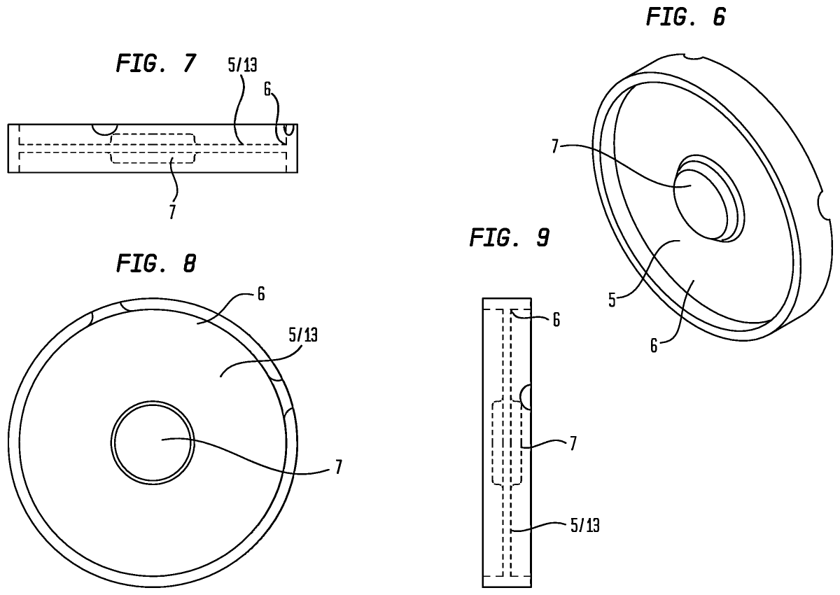

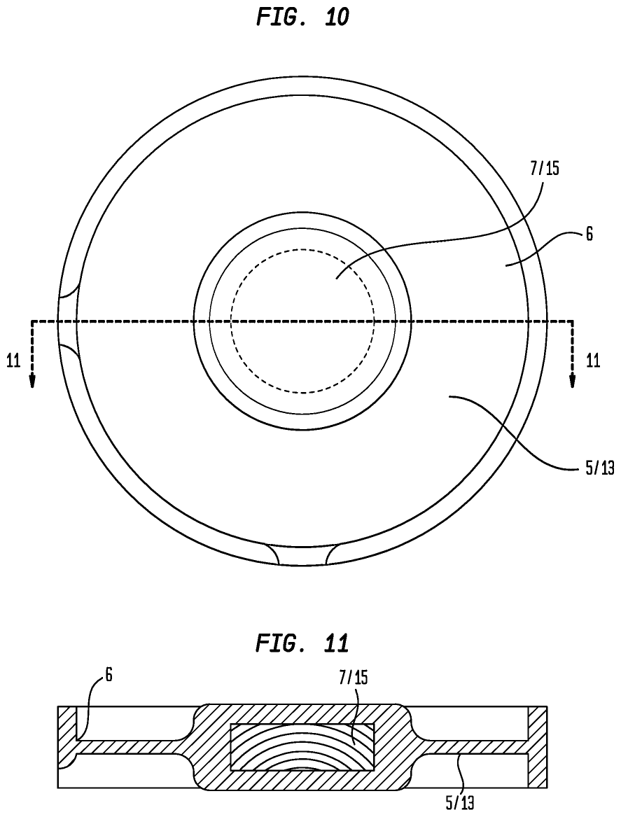

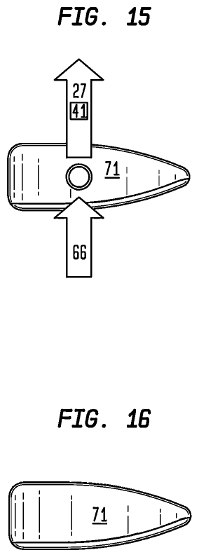

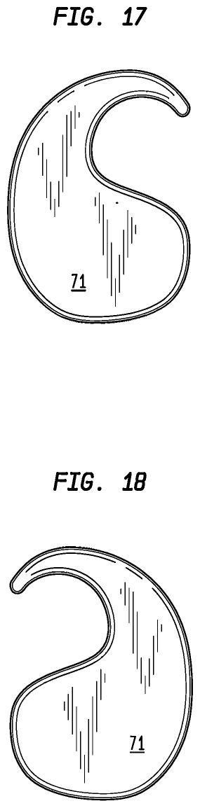

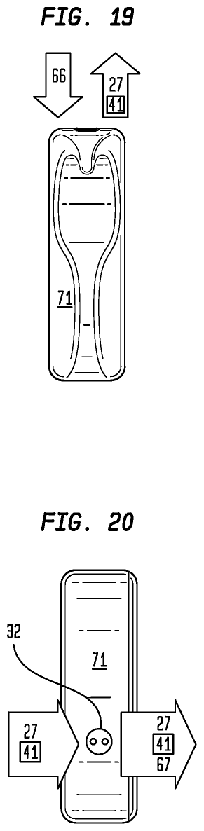

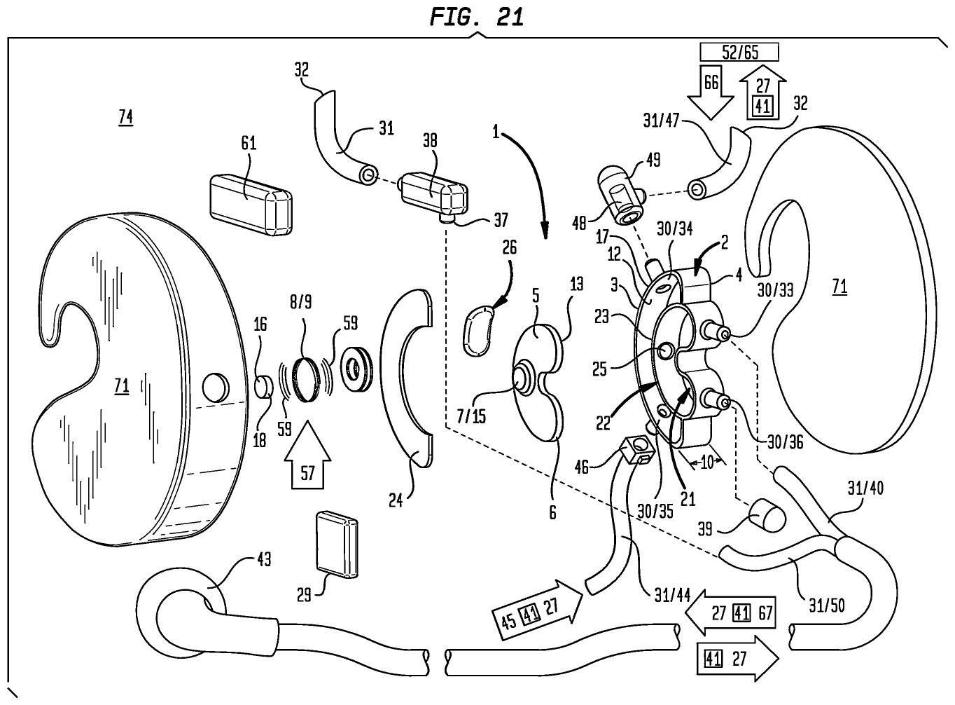

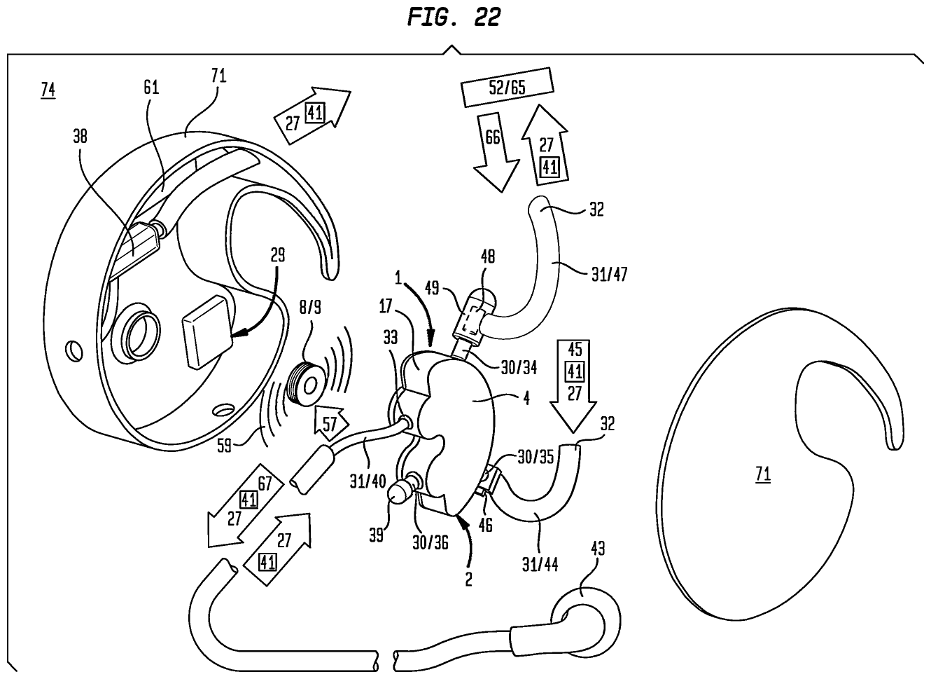

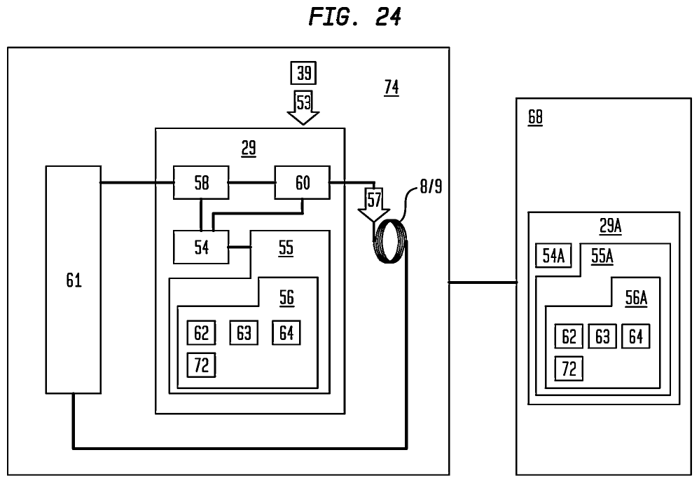

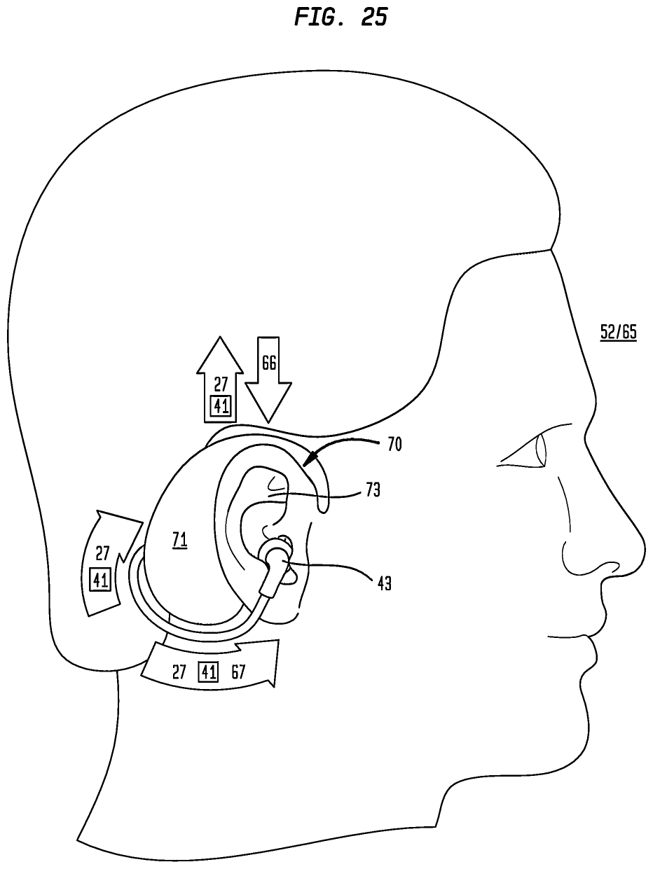

Referring generally to FIGS. 1 through 25, particular embodiments of a magnetically driven pressure generator (1) can include one or more of: a housing (2) having an open end (3) and a closed end (4), a flexible member (5) having a peripheral margin (6) sealably coupled to the open end (3) of said housing (2), a first magnetic force generator (7) disposed on the flexible member (5), and a second magnetic force generator (8) disposed proximally adjacent the flexible member (5), where either the first magnetic force generator (7) or second magnetic force generator (8) comprises an electromagnetic force generator (9).

Now referring primarily to FIGS. 1 and 21, the housing (2) can have a housing depth (10) disposed between an open end (3) and a closed end (4). In particular embodiments, the housing (2) can be configured as a right cylinder (as shown in the illustrative example of FIGS. 1 through 5); however, this is not intended to preclude embodiments which otherwise include the housing (2) having a configuration in cross section, such as a rectangle, square, triangle, elliptical cylinder, or combinations thereof, or where the sides are arcuate or have an amount of curvature and are not linear. The housing (2) can be composed of a substantially non-electrically conducting, rigid material, such as plastic, rubber, elastomer, glass, ceramics, or the like. The housing (2) can be fabricated, molded, or formed from a plurality of pieces or as one-piece. As illustrative examples: sintering of metal powders, plastic injection molding, waterjet machining, or combinations thereof.

Now referring primarily to FIGS. 1 through 13, a flexible member (5) can be sealably engaged along the peripheral margin (6) proximate the open end (3) of the housing (2). The term “sealably engaged” means engagement of the peripheral margin (6) of the flexible member (5) at or proximate to the open end (3) of the housing (2) to effect a substantially fluid tight seal, and without limitation to the breadth of the foregoing, includes as illustrative examples, a substantially fluid tight seal effected by compression between surfaces of the peripheral margin (6) of the flexible member (5) at or proximate to the open end (3) of the housing (2), adhesive applied between the surfaces of the peripheral margin (6) of the flexible member (5) and the open end (3) of the housing (2), laser welding of the peripheral margin (6) of the flexible member (5) to the open end (3) of the housing (2), or combinations thereof. The flexible member (5) can comprise one or more of a substantially non-electrically conductive elastomer, thermoplastic, or other material that can flex or deform from a first position to a second position, resiliently or non-resiliently, to correspondingly increase or decrease the volume of the enclosed space (11) in the housing (2). Additionally, the flexible member (5) can be substantially fluid impermeable or partially fluid impermeable during the normal operating cycle of the magnetically driven pressure generator (1). The flexible member (5) can be disposed at or proximate the open end (3) of the housing (2) to define an enclosed space (11) inside the housing (2), the enclosed space (11) bound by the internal surface (12) of the housing (2) and a first side (13) of the flexible member (5).

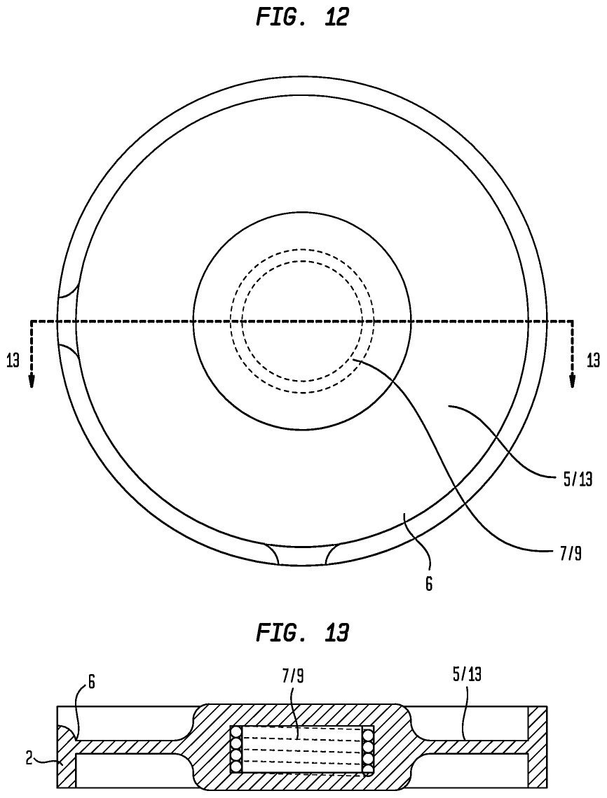

Again, referring primarily to FIGS. 1 through 13, a first magnetic force generator (7) can be disposed on or in the flexible member (5), and a second magnetic force generator (8) can be disposed axially adjacent to the first magnetic force generator (7) and proximate to the closed end (4) or proximate the open end (3) of the housing (2), whether disposed in the enclosed space (11) or disposed external to the housing (2) (as shown in the illustrative example of FIGS. 1-5). As to particular embodiments, the first magnetic force generator (7) can be an electromagnetic force generator (9) and the second magnetic force generator (8) can be a magnet (15)(as shown in the illustrative example of FIG. 13), or the first magnetic force generator (7) can be a magnet (15) and the second magnetic force generator (8) can be an electromagnetic force generator (9)(as shown in the illustrative examples of FIGS. 1 through 11). The term “magnet” means a material that retains its magnetic properties in the absence of an inducing field or current and, without limitation to the breadth of the foregoing, can be a piece of metal surrounded by a magnetic field which can be aligned with, attracted to, or repelled by an external magnetic field, and as illustrative examples: neodynium iron boron, samarium cobalt, alnico, ceramic or ferrite, or the like. The term “electromagnetic force generator” means an electrically conductive winding of a conductive material which upon passage of an electrical current generates a magnetic field (59) and, without limitation to the breadth of the foregoing, can as illustrative examples be one or more electrically conductive windings of: copper, silver, brass, or other like conductive materials or combinations thereof.

Again, referring primarily to FIGS. 1 through 5, particular embodiments can, but need not necessarily, include a first ferromagnetic core (16). The term “ferromagnetic core” means a body susceptible to magnetization in an applied electromagnetic field and, without limitation to the breadth of the foregoing, can be a one-piece body or a body comprising a plurality of layers of material susceptible to magnetization, such as: nickel, iron, cobalt, or other like material, or combinations thereof. In particular embodiments, the first magnetic force generator (7) coupled to the flexible member (5) can be a magnet (15) and the second magnetic force generator (8) can be an electromagnetic force generator (9) with the first ferromagnetic core (16) having a location responsive to the second magnetic force generator (8). In particular embodiments, the first ferromagnetic core (16) can be generally axially aligned with the first magnetic force generator (7). As to particular embodiments, the electromagnetic force generator (9) can engage the first ferromagnetic core (16) or be disposed a spaced distance about the first ferromagnetic core (16). As to particular embodiments, the second magnetic force generator (8) can comprise a plurality of metal windings wound about the external surface (17) of the housing (2) proximate to the closed end (4), and the first ferromagnetic core (16) can be disposed a spaced distance apart within the electromagnetic force generator (9) axially aligned with the first magnetic force generator (7)(as shown in the illustrative example of FIG. 5). In particular embodiments, the second magnetic force generator (8) can comprise a plurality of windings wound about the ferromagnetic core external surface (18) with the first ferromagnetic core (16) substantially axially aligned with the first magnetic force generator (7).

Now referring primarily to FIGS. 1 through 5, particular embodiments can, but need not necessarily, include a third magnetic force generator (19), which can be disposed at or proximate to the open end (3) or the closed end (4) of the housing (2). The third magnetic force generator (19) can be a magnet (15) or electromagnetic force generator (9), as described above. As to embodiments that include a third magnetic force generator (19) in the form of an electromagnetic force generator (9), a second ferromagnetic core (20) can, but need not necessarily, be disposed proximate to the open end (3) of the housing (2) at a location to which the second ferromagnetic core (20) can be responsive to the electromagnetic field applied by the electromagnetic force generator (9). The second ferromagnetic core (20) can be disposed to generally axially align with the first magnetic force generator (7) disposed on the flexible member (5).

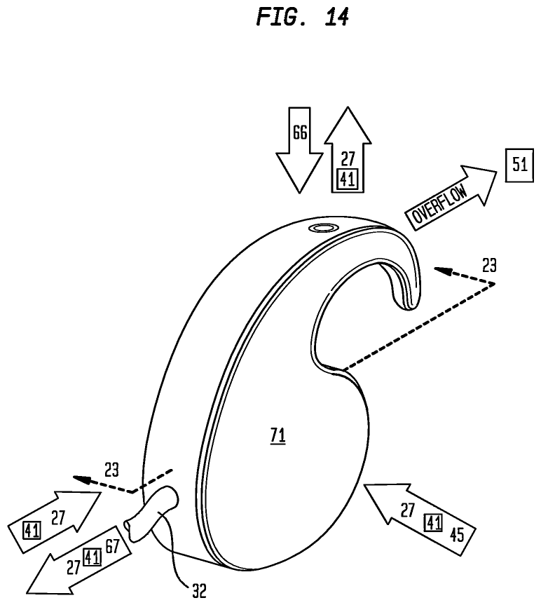

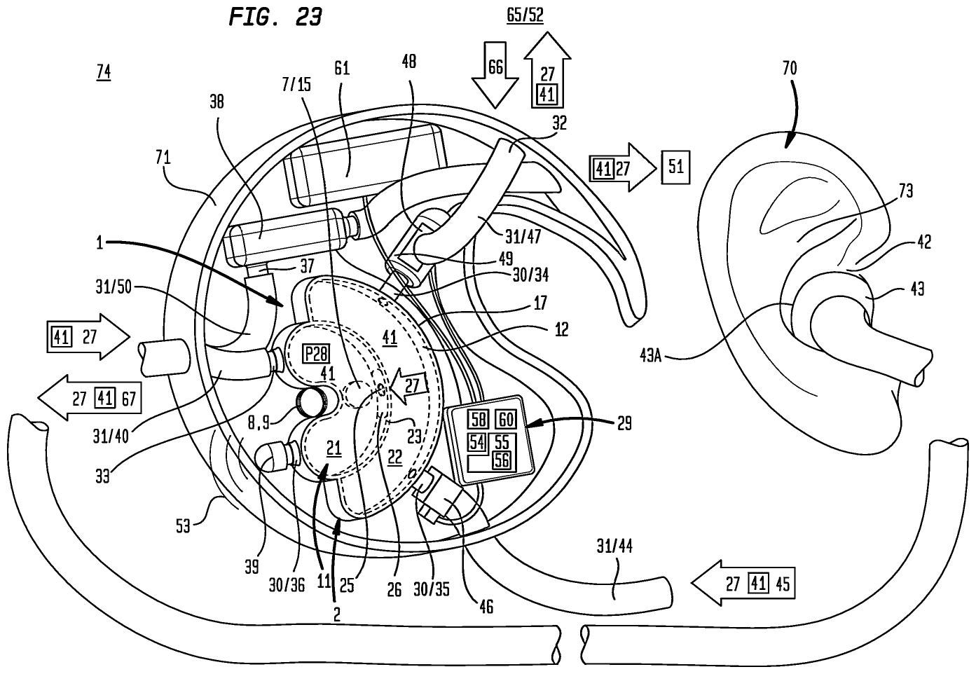

Now referring primarily to FIGS. 14 through 23, in particular embodiments, the housing (2) can, but need not necessarily, be partitioned into a first fluid chamber (21) and a second fluid chamber (22) by a partition wall (23). The partition wall (23) can be sealably engaged with the closed end (4) of the housing (2) and extend to the open end (3) of the housing (2). The flexible member (5) can be sealably engaged along the peripheral margin (6) at or proximate to the open end (3) of the first fluid chamber (21). A cover (24) can be sealably engaged to or proximate the open end (3) of the second fluid chamber (22). The cover (24) can be comprised of one or more substantially non-electrically conductive elastomers, thermoplastics, or the like. Additionally, the cover (24) can be substantially fluid impermeable during the normal operating cycle of the magnetically driven pressure generator (1). The partition wall (23) can further include an aperture (25) communicating between the first fluid chamber (21) and second fluid chamber (22). A first unidirectional valve (26) can be disposed in the partition wall (23) to permit a fluid flow (27) to move in only one direction between the first and second chambers (21)(22). The first unidirectional valve (26) disposed in the partition wall (23) can be responsive to negative or positive fluid pressure (28) within the first or second fluid chambers (21)(22) (as shown in the illustrative example of FIGS. 21 and 23, the first unidirectional valve (26) can comprise a flap valve) or the first unidirectional valve (26) can be an electrically operable between an open condition and a closed condition in response to a signal from a controller (29), as further described below.

Again, referring primarily to FIGS. 1 through 23, particular embodiments can, but need not necessarily, further include one or more ports (30) disposed in the housing (2) which communicate between the internal surface (12) and external surface (17) of the housing (2). Particular embodiments can, but need not necessarily, further include conduits (31) engaged to the one or more ports (30) to extend the enclosed space (11) of the housing (2) to the distal end (32) of the conduits (31) allowing an increase or decrease of fluid pressure (28) or fluid flow (27) within the conduits (31) at the distal end (23). As to particular embodiments, the housing (2) can include one or more of a fluid outlet port (33), a fluid bleed port (34), a fluid inlet port (35), and a pressure sensor port (36). The fluid outlet port (33) can communicate between the external surface (17) and the internal surface (12) of the housing (2) to provide a fluid flow (27) from the enclosed space (11)(as to the illustrative embodiments of FIGS. 1-13) or from the first fluid chamber (21) (as shown in the illustrative embodiments of FIGS. 14-23). A fluid delivery conduit (40) can be sealably engaged to the fluid outlet port (33) for delivery of an amount of fluid (41) to the distal end (32) of the fluid delivery conduit (40), which can, but need not necessarily, be disposed to deliver an amount of fluid (41) from the enclosed space (11) of the housing (2) or the first chamber (21).

Now referring primarily to FIGS. 14-23, a fluid inlet port (35) can communicate between the external surface (17) and the internal surface (12) of the housing (2) of the second chamber (22). The fluid inlet port (35) can sealably engage a fluid inlet conduit (44) open to atmosphere in the ambient environment (65) or coupled to a fluid source (45) which can contain an amount of fluid (41). The amount of fluid (41) contained by the fluid source (45) can be, as illustrative examples: a liquid, a gel, a viscous polymer, or other material, or combinations thereof, which deforms continuously for delivery from the fluid source (45) into the second fluid chamber (22). The amount of fluid (41) contained in the fluid source (45) can be delivered as a fluid flow (27) from the fluid source (45) under force of one or more of: gravity, pressurized head space, a fluid pump, or combinations thereof. As to particular embodiments, a fluid inlet valve (46) can be disposed between the fluid inlet port (35) and the fluid source (45) to intermittently or continuously interrupt flow of an amount of fluid (41) to or from the second fluid chamber (22) toward the fluid source (45).

Again, referring primarily to FIGS. 14 through 23, the fluid bleed port (34) can communicate between the external surface (17) and internal surface (12) of the housing (2) of the second chamber (22). The fluid bleed port (34) can sealably engage a fluid bleed conduit (47) and the distal end (32) of the fluid bleed conduit (47) can be disposed in the ambient environment (65). The fluid bleed conduit (43) can conduct an amount of fluid (41) from the second chamber (22) to the ambient environment (65). If the amount of fluid (41) delivered from the fluid source (45) exceeds the volume of the second fluid chamber (22), the excess amount of fluid (38) can egress from the second fluid chamber (22) through the fluid bleed port (34) and the fluid bleed conduit (47) to the ambient environment (65).

Again, referring primarily to FIGS. 14 through 23, as to particular embodiments, a fluid flow generator (49) can be coupled between the fluid bleed port (35) and the distal end (32) of the fluid bleed conduit (46). The fluid flow generator (49) can operate in the first instance to provide the bleed valve (48) in the open condition, which allows egress of an excess amount of fluid (41) from the second fluid chamber (22). The fluid flow generator (49) in the second instance can operate to generate a flow of air (66) from the ambient environment (65) into the second fluid chamber (22) to move the amount of fluid (41) contained in the second fluid chamber (22) through the aperture (25) disposed in the partition wall (23) into the first fluid chamber (21).

Now referring primarily to FIGS. 1 through 23, embodiments can, but need not necessarily, further include a fluid pressure relief valve (38). As to particular embodiments, the fluid pressure relief valve (38) can be coupled to a pressure relief port (37) which communicates between the external surface (17) and the internal surface (12) of the housing (2) of the enclosed space (11)(as shown in the illustrative example of FIGS. 1 through 5). As to other embodiments, a fluid return conduit (50) can, but need not necessarily, be fluidicly coupled to the fluid delivery conduit (40) to return the amount of fluid (41) to the fluid source (45), a fluid collection vessel (51), or discharge the amount of fluid to the ambient environment (65) (as shown in the illustrative examples of FIGS. 14 through 23). As to these embodiments the pressure relief valve (38) can operate between a closed condition to generate an amount of fluid pressure (28) in the first fluid chamber (21), the fluid delivery conduit (40), or the fluid return conduit (50), and an open condition to relieve an amount of fluid pressure (28) in the first fluid chamber (21), the fluid delivery conduit (40), or the fluid return conduit (50).

As illustrative examples, the pressure release valve (38) can be configured to relieve an amount of pressure (28) in the enclosed space (11) or first fluid chamber (21) when the amount of pressure (28) exceeds a pre-selected pressure (28) to actuate the pressure release valve (38). In one illustrative embodiment, the pressure release valve (38) can be disposed in the open condition when the amount of pressure (28) exceeds 5.0 psi (pounds per square inch; about 34 kPa; 1 psi=about 6.8 kPa). As to particular embodiments, the pressure release valve (38) can be disposed in the open condition in response to lesser or greater amounts of pressure (28) in a range of about 0 psi to 20 psi (about 0 kPa to about 137.8 kPa). The amount of pressure (28) can be selected from the group including or consisting of: about 0.0 psi to about 1.0 psi, about 0.5 psi to about 1.5 psi, about 1.0 psi to about 2.0 psi, about 1.5 psi to about 2.5 psi, about 2.0 psi to about 3.0 psi, about 2.5 psi to about 3.5 psi, about 3.0 psi to about 4.0 psi, about 3.5 psi to about 4.5 psi, about 4.0 psi to about 5.0 psi, about 4.5 psi to about 5.5 psi, about 5.0 psi to about 6.0 psi, about 5.5 psi to about 6.5 psi, about 6.0 psi to about 7.0 psi, about 6.5 psi to about 7.5 psi, about 7.0 psi to about 8.0 psi, about 7.5 psi to about 8.5 psi, about 8.0 psi to about 9.0 psi, about 8.5 psi to about 9.5 psi, about 9.0 psi to about 10.0 psi, about 9.5 psi to about 10.5 psi, about 10.0 psi to about 11.0 psi, about 10.5 psi to about 11.5 psi, about 11.0 psi to about 12.0 psi, about 11.5 psi to about 12.5 psi, about 12.0 psi to about 13.0 psi, about 12.5 psi to about 13.5 psi, about 13.0 psi to about 14.0 psi, about 13.5 psi to about 14.5 psi, about 14.0 psi to about 15.0 psi, about 14.5 psi to about 15.5 psi, about 15.0 psi to about 16.0 psi, about 15.5 psi to about 16.5 psi, about 16.0 psi to about 17.0 psi, about 16.5 psi to about 17.5 psi, about 17.0 psi to about 18.0 psi, about 17.5 psi to about 18.5 psi, about 18.0 psi to about 19.0 psi, about 18.5 psi to about 19.5 psi, and about 19.0 psi to about 20.0 psi.

The foregoing embodiments are not intended to preclude embodiments which dispose the pressure release valve (24) in the open condition at a fluid pressure (28) of greater than 20 psi, depending on the application.

Now referring primarily to FIGS. 1 through 23, particular embodiments can, but need not necessarily, further include a pressure sensor (39). The pressure sensor (39) can be fluidicly coupled to the enclosed space (11) of the housing (2) or first fluid chamber (21) to sense the amount of pressure (28) inside the enclosed space (11) or first fluid chamber (21). The pressure sensor (39) can generate a signal (53) which varies based on the increase or decrease of pressure (28) within the enclosed space (11) or first fluid chamber (21) within the housing (2).

Now referring generally to FIGS. 1 through 24, with particular reference to FIGS. 21 through 24, particular embodiments can, but need not necessarily, further include a controller (29) including a controller processor (54) communicatively coupled to a controller non-transitory computer readable media (55) containing a computer program (56) executable by the controller processor (54) to control the direction and magnitude of current (57) in the one or more electromagnetic force generators (9). The controller (29) can be contained inside of the magnetically driven pressure generator (1) or within a casing (71) enclosing the magnetically driven pressure generator (1) or can be electronically coupled (whether wired or wirelessly) through intermediary hardware to an external controller (29A) in which the processor (54A) the non-transitory computer readable medium (55A) containing the computer program (56A) resides in a mobile device (68), such as: a cellular telephone, tablet computer, laptop computer, or other computer implemented device in which the computer program (56A) can reside.

As to particular embodiments, the computer program (56)(56A) can operate a current controller (58) electrically coupled to one or more electromagnetic force generators (9)(19). The current controller (58) can function to control the magnitude of the current (57) conducted through the one or more electromagnetic force generators (9)(19). The current controller (58) can be adapted for use with alternating current, direct current, or both. The magnetic field (59) generated by the electromagnetic force generator (9) can be proportional to the magnitude of the current (57). Accordingly, the current controller (58), by varying the amplitude of the current (57), can correspondingly continuously or intermittently vary the strength of the magnetic field (59) to correspondingly continuously control flexure of the flexible member (5) to intermittently or continuously precisely form pressure waves (67) having pre-selected amplitude and frequency values (63)(64) over time.

As to particular embodiments, the computer program (56) can further operate a polarity controller (60) electrically coupled to the one or more electromagnetic force generators (9). The polarity controller (60) operates to control the direction of the current (57) being conducted through the one or more electromagnetic force generators (9)(19). The direction of magnetic polarity generated by the electromagnetic force generator (9) can be changed by correspondingly changing the direction of current (57) flowing in the electromagnetic force generator (9)(19). Accordingly, the polarity controller (60) can, by changing the direction of the current (57) in the electromagnetic force generator, (9)(19) correspondingly change the direction of the magnetic polarity generated by the electromagnetic force generator (9)(19).

Particular embodiments can further include a power source (61). The power source (61) can be electrically coupled to the one or more electromagnetic force generators (9) directly, through intermediary hardware (the microprocessor, a current controller, a polarity controller), or both. Further, the power source (61) can provide power convertible to alternating current, direct current, or both.

Now referring primarily to FIGS. 1 through 23, particular embodiments of the magnetically driven pressure generator (1) can be used to generate either an increase or decrease in pressure (28) of a fluid flow (27) of an amount of fluid (41) in or from the enclosed space (11) or the first fluid chamber (21) depending on the embodiment. As an illustrative example, referring to FIGS. 1 through 13, the magnetically induced pressure generator (1) can be configured to operate the second magnetic force generator (8) to induce an amount of flexure in the flexible member (5) to correspondingly alter the volume of the enclosed space (11) to correspondingly increase or decrease pressure of an amount of fluid (41) contained therein. A gas contained in a closed system, exhibits an inverse relationship between pressure and volume. Accordingly, if the flexible member (5) flexes toward the closed end (4) of the housing (2), the volume of the enclosed space (11) correspondingly decreases, and the fluid pressure (28) of the gas within the enclosed space (11) can correspondingly increase. Conversely, if the flexible member (5) flexes away from the closed end (4) of the housing (2), the volume of the enclosed space (11) correspondingly increases, and the pressure of the gas within the enclosed space (11) correspondingly decreases. In the aforementioned particular embodiments, the amplitude of change in pressure (28) of the gas in the enclosed space (11) can be proportionate to the amount of flexure of the flexible member (5) induced by attracting or repulsing forces generated between the first magnetic force generator (7) and the second magnetic force generator (8). Additionally, alternating the attracting and repulsing forces generated between the first magnetic force generator (7) and the second magnetic force generator (8) can correspondingly generate oscillation in the flexible member (5) in an oscillation period independent of the oscillation amplitude. Accordingly, pressure waves can be precisely generated in the enclosed space (11) of the housing (2) having a pre-selected amplitude or frequency values (63)(64) over a period of time by operation of the current controller (58) and the polarity controller (60).

Referring primarily to FIGS. 14 through 23, particular embodiments of the magnetically driven pressure generator (1) can operate to alter fluid pressure (28) or generate a fluid flow (27) in an amount of fluid (41), whether the fluid is a liquid or a gas. As an illustrative example, the enclosed space (11) can, as above described, include a first fluid chamber (21), a second chamber (22), a fluid delivery conduit (40), a fluid return conduit (50), and an earpiece (43) coupled to the fluid delivery conduit (40) and the fluid return conduit (50) which can be disposed in or sealably engaged to the external ear canal (42). The fluid inlet valve (46) can be disposed in the open condition to allow an amount of fluid (41) to be delivered from the fluid source (45) through the fluid inlet conduit (44) to the second fluid chamber (22). As an amount of fluid (41) flows into the second fluid chamber (22), the bleed valve (48) within the fluid flow generator (49) can be disposed in the open condition to permit air inside the second fluid chamber (22) to flow through the fluid bleed conduit (47) and to the ambient environment (65). Once the second fluid chamber (22) contains an amount of fluid (22), the fluid flow generator (49) can be further operated to generate a flow of air (67) into the second fluid chamber (22) to force the amount of fluid (41) within the second fluid chamber (22) through the aperture (25) disposed in the partition wall (23) into the first fluid chamber (21). The first unidirectional valve (26) operates to prohibit fluid flow (27) from the first fluid chamber (21) back into the second fluid chamber (22). By operation of the flexible member (5) the amount of fluid (41) can then be delivered from the first fluid chamber (21) through the fluid delivery conduit (40) and through the fluid return conduit (50). As one illustrative example, the fluid delivery conduit (40) and the fluid return conduit (50) can be disposed in the external ear canal (42) of an ear (70), and as to certain embodiments, the fluid delivery conduit (40) and the fluid return conduit (50) can pass through or be surrounded by an earpiece (43) which can be disposed in or sealably engaged with the external ear canal (42) of the ear (70). The amount of fluid (41) can be delivered into the external ear canal (42) from the fluid delivery conduit (40), circulate in the external ear canal (42), pass into the fluid return conduit (50), and through the pressure release valve (38) in the open condition. The pressure relief valve (38) can then be disposed in the closed condition to allow the pre-selected fluid pressure (28) to be generated in the fluid delivery conduit (40), the fluid return conduit (50), and in the external ear canal (42) of the ear (70) when the earpiece (43) engages or sealably engages the external ear canal (42).

As above described, operation of the first magnetic force generator (7) and the second magnetic force generator (8) can effect an amount of flexure in the flexible member (5) to correspondingly alter the volume of the first fluid chamber (21) to correspondingly increase or decrease fluid pressure of the fluid (41) therein. The flexure of the flexible member (5) toward the closed end (4) of the housing (2) can decrease the volume of the first fluid chamber (21), without substantially increasing or decreasing the surface area of the first fluid chamber (21) or volume of amount of fluid (38) within the first fluid chamber (21), thereby increasing the fluid pressure (28) within the first fluid chamber (21). The flexure of the flexible member (5) can also occur away from the closed end (4) of the housing (2), which increases the volume of the first fluid chamber (21) without substantially increasing or decreasing the surface area of the first fluid chamber (21) or volume of amount of fluid (41) within the first fluid chamber (21), thereby decreasing the fluid pressure (28) within the first fluid chamber (21). In the aforementioned particular embodiments, the amplitude of change in fluid pressure (28) of the amount of fluid (41) in the first fluid chamber (21) can be proportionate to the amount of flexure of the flexible member (5) induced by attracting or repulsing forces generated between the first magnetic force generator (7) and the second magnetic force generator (8). Additionally, alternating the attracting and repulsing forces generated between the first magnetic force generator (7) and the second magnetic force generator (8) can correspondingly generate oscillation in the flexible member (5) in an oscillation period independent of the oscillation amplitude. Accordingly, pressure waves (67) can be generated in the first fluid chamber (21) having a pre-selected amplitude (63) and frequency values (64) by operation of the current controller (58) and the polarity controller (60).

Again, referring to FIG. 5, in particular embodiments, a third magnetic force generator (19) can be included comprising either a magnet (15) or electromagnetic force generator (9) to further interact with the attracting forces or repulsing forces of the first magnetic force generator (7) and second magnetic force generator (8), flexing the flexible member (5) accordingly, as described above.

In a particular embodiment, the operation of the program (56) can be executed to oscillate the flexible member (5) as described above, at a pre-selected oscillation frequency (64). The oscillation frequency (64) can be in a range of about 0 to about 100 kiloHertz (kHz). The oscillation frequency can be selected from the group including or consisting of: about 0 kHz to about 5.0 kHz, about 2.5 kHz to about 7.5 kHz, about 5.0 kHz to about 10.0 kHz, about 7.5 kHz to about 12.5 kHz, about 10.0 kHz to about 15.0 kHz, about 12.5 kHz to about 17.5 kHz, about 15.0 kHz to about 20.0 kHz, about 17.5 kHz to about 22.5 kHz, about 20.0 kHz to about 25.0 kHz, about 22.5 kHz to about 27.5 kHz, about 25.0 kHz to about 30.0 kHz, about 27.5 kHz to about 32.5 kHz, about 30.0 kHz to about 35.0 kHz, about 32.5 kHz to about 37.5 kHz, about 35.0 kHz to about 40.0 kHz, about 37.5 kHz to about 42.5 kHz, about 40.0 kHz to about 45.0 kHz, about 42.5 kHz to about 47.5 kHz, about 45.0 kHz to about 50.0 kHz, about 47.5 kHz to about 52.2 kHz, about 50.0 kHz to about 55.0 kHz, about 52.5 kHz to about 57.5 kHz, about 55.0 kHz to about 60.0 kHz, about 57.5 kHz to about 62.5 kHz, about 60.0 kHz to about 65.0 kHz, about 62.5 kHz to about 67.5 kHz, about 65.0 kHz to about 70.0 kHz, about 67.5 kHz to about 72.5 kHz, about 70.0 kHz to about 75.0 kHz, about 72.5 kHz to about 77.5 kHz, about 75.0 kHz to about 80.0 kHz, about 77.5 kHz to about 82.5 kHz, about 80.0 kHz to about 85.0 kHz, about 82.5 kHz to about 87.5 kHz, about 85.0 kHz to about 90.0 kHz, about 87.5 kHz to about 92.5 kHz, about 90.0 kHz to about 95.0 kHz, about 92.5 kHz to about 97.5 kHz, about 95.0 kHz to about 100 kHz, and combinations thereof.

In yet another particular embodiment, the computer program (56) can be executed to generate a pre-selected pressure amplitude (63), whether a positive pressure or negative pressure as compared to the ambient pressure (52) in the enclosed space (11) or first fluid chamber (21), depending upon the embodiment and application. In one illustrative embodiment, the range of pre-selected pressure amplitude (63) can be about 0 psi to about 5 psi (about 0 kPa to about 34.4 kPa; 1 psi=6.8 kPa). In another illustrative embodiment, the pre-selected pressure amplitude (63) can be a range of pressures of about 0 psi to about 20 psi (about 0 kPa to about 137.8 kPa; 1 psi=6.8 kPa). The pre-selected pressure amplitude (63) in the closed space (11) or the first fluid pressure chamber (21) can be selected from the group including or consisting of: about 0.0 psi to about 1.0 psi, about 0.5 psi to about 1.5 psi, about 1.0 psi to about 2.0 psi, about 1.5 psi to about 2.5 psi, about 2.0 psi to about 3.0 psi, about 2.5 psi to about 3.5 psi, about 3.0 psi to about 4.0 psi, about 3.5 psi to about 4.5 psi, about 4.0 psi to about 5.0 psi, about 4.5 psi to about 5.5 psi, about 5.0 psi to about 6.0 psi, about 5.5 psi to about 6.5 psi, about 6.0 psi to about 7.0 psi, about 6.5 psi to about 7.5 psi, about 7.0 psi to about 8.0 psi, about 7.5 psi to about 8.5 psi, about 8.0 psi to about 9.0 psi, about 8.5 psi to about 9.5 psi, about 9.0 psi to about 10.0 psi, about 9.5 psi to about 10.5 psi, about 10.0 psi to about 11.0 psi, about 10.5 psi to about 11.5 psi, about 11.0 psi to about 12.0 psi, about 11.5 psi to about 12.5 psi, about 12.0 psi to about 13.0 psi, about 12.5 psi to about 13.5 psi, about 13.0 psi to about 14.0 psi, about 13.5 psi to about 14.5 psi, about 14.0 psi to about 15.0 psi, about 14.5 psi to about 15.5 psi, about 15.0 psi to about 16.0 psi, about 15.5 psi to about 16.5 psi, about 16.0 psi to about 17.0 psi, about 16.5 psi to about 17.5 psi, about 17.0 psi to about 18.0 psi, about 17.5 psi to about 18.5 psi, about 18.0 psi to about 19.0 psi, about 18.5 psi to about 19.5 psi, about 19.0 psi to about 20.0 psi, and combinations thereof. The above embodiments are illustrative only, as the pre-selected pressure amplitude can be selected from the range of about 0 psi to about any pre-selected pressure amplitude, depending on the application.

By combining pre-selected pressure amplitudes (63) in various combinations and permutations with pre-selected oscillation frequencies (64) over a period of time, stable pressure amplitudes (63)(whether positive or negative relative to the ambient pressure (52)) or pressure waves (67) having preselected amplitude values (63) or frequency values (64), or combinations thereof, can be generated in the enclosed space (11) or the first fluid pressure chamber (21) to track pre-selected pressure profiles (62) of the program (56). To ensure that the pre-selected amplitude and frequency values (63)(64) or pre-selected pressure profiles (62) are achieved, the pressure sensor signal (53) generated by the pressure sensor (39) coupled to the pressure sensor port (36) can be analyzed by a feedback module (72) of the computer program (56) to correspondingly alter operation of the flexible member (5).

Now referring primarily to FIGS. 21 through 25, as to particular embodiments, the distal ends (32) of the fluid delivery conduit (40) or the fluid return conduit (50) can be disposed in the external ear canal (42) of an ear (70). An amount of fluid (41) can be delivered from the first fluid chamber (21) of the housing (2) to the distal end (32) of the fluid delivery conduit (40) into the external ear canal (42) of an ear (70) and egress the external ear canal (42) of the ear (70) through the fluid return conduit (50). The pressure relief valve (38) can be disposed in the open condition or closed condition depending on the amount of fluid pressure (28) to be generated in the external ear canal (42) of the ear (70). As to particular embodiments, the pressure relief valve (38) can be maintained in the open condition to allow an amount of fluid (41) to circulate in the external ear canal (42) of the ear (70) at a relatively low fluid pressure (28). As to other embodiments, the pressure relief valve (38) can be maintained in the closed condition or intermittently closed condition to generate a continuous or substantially continuous fluid pressure (28)(whether negative or positive compared to ambient pressure (52)) or a variable fluid pressure (28), or a fluid pressure adjusted over a period time to track a pre-selected pressure profile (62)(a set of pre-selected pressure values over a period of time).

Again referring primarily to FIGS. 14 through 25, embodiments can further include a casing (71) which encloses or operably supports one or more of: the housing (2), the magnetic force generators (8)(9)(19), ferromagnetic cores (16)(20), fluid flow generator (49), controller (29), conduits (31), and associated circuitry. The casing (71), as to particular embodiments, can be configured to couple, connect, attach, or bring embodiments of the magnetically driven pressure generator (1) into proximity with an object or person for use. As shown in the illustrative example of FIG. 25, the casing (71) can be configured to provide an external ear canal pressure regulation device (74) which can be disposed behind and about the auricle (73) of the ear (70).

As can be easily understood from the foregoing, the basic concepts of the present invention may be embodied in a variety of ways. The invention involves numerous and varied embodiments of a magnetically driven pressure generator and methods for making and using such a magnetically driven pressure generator including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “electromagnetic force generator” should be understood to encompass disclosure of the act of “generating an electromagnetic force”—whether explicitly discussed or not—and, conversely, were there effectively disclosure of the act of “generating an electromagnetic force”, such a disclosure should be understood to encompass disclosure of an “electromagnetic force generator” and even a “means for generating an electromagnetic force.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the magnetically driven pressure generator herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. (canceled)

2. A device comprising:
a housing at least partially defining a chamber;
a flexible member configured to flex to increase or decrease a volume of said chamber;
a first magnetic force generator disposed on said flexible member;
a second magnetic force generator disposed on said housing, said first magnetic force generator being responsive to said second magnetic force generator to flex said flexible member, at least one of said first magnetic force generator or said second magnetic force generator comprising an electromagnetic force generator being configured to flex said flexible member to increase or decrease said volume of said chamber;
a fluid delivery conduit fluidically coupled to the chamber and operable to conduct fluid into or out of the chamber; and
an earpiece fluidically coupled to said fluid delivery conduit.

3. The device of claim 2, wherein the device is an ear canal pressure regulation device configured to control pressure in an external ear canal.

4. The device of claim 3, wherein the device is configured to produce a positive pressure and a negative pressure in the external ear canal as compared to an ambient pressure.

5. The device of claim 2, comprising a ferromagnetic core responsive to said second magnetic force generator, said ferromagnetic core generally axially aligned with said first magnetic force generator disposed on said flexible member.

6. The device of claim 2, wherein said electromagnetic force generator is operable to intermittently flex said flexible member to increase or decrease said volume of said chamber, said electromagnetic force generator operates to oscillate said flexible member at a frequency of 0 kHz to 100 kHz.

7. The device of claim 6, wherein said chamber has a closed condition, and wherein flexing said flexible member to increase or decrease said volume of said chamber generates an amount of pressure in said chamber.

8. The device of claim 7, wherein said amount of pressure comprises a positive amount of pressure or a negative amount of pressure.

9. The device of claim 8, wherein said amount of pressure comprises 0 psi to 20 psi.

10. The device of claim 2, comprising a port communicating between opposite internal and external surfaces of said housing, wherein the fluid delivery conduit is coupled to the port.

11. The device of claim 2, comprising a pressure relief port coupled to a fluid pressure relief valve operable to release pressure from the chamber.

12. The device of claim 11, wherein said fluid pressure release valve operable to release pressure above a threshold of at least 20 psi.

13. The device of claim 2, comprising a third magnetic force generator disposed on said housing, said first magnetic force generator responsive to said second magnetic force generator or said third magnetic force generator to flex said flexible member.

14. The device of claim 13, comprising a ferromagnetic core responsive to said third magnetic force generator, said ferromagnetic core generally axially aligned with said first magnetic force generator disposed on said flexible member.

15. The device of claim 13, wherein the first magnetic force generator is disposed between the second magnetic force generator and the third magnetic force generator.

16. The device of claim 3, further comprising:
a fluid return conduit coupled to said earpiece to permit fluid to flow from said external ear canal into the earpiece.

17. The device of claim 16, comprising a pressure release valve coupled to said fluid return conduit and operable between an open condition which allows a flow of said fluid from said external ear canal and a closed condition which interrupts said flow of said fluid from said external ear canal.

18. The device of claim 16, comprising a fluid outlet port communicating between opposite internal and external surfaces of said housing, wherein the fluid delivery conduit is coupled to the outlet port, and a fluid inlet port communicating between said external surface and said internal surface of the housing, wherein the fluid return conduit is coupled to the inlet port.

19. The device of claim 2, comprising:
a fluid inlet port; and
a fluid source coupled to a fluid inlet conduit, said fluid source comprising fluid.

20. The device of claim 19, further comprising a fluid inlet valve disposed between said fluid inlet port and the fluid source, said fluid inlet valve being configured to interrupt fluid flow to said housing.

21. The device of claim 20, further comprising a fluid bleed port communicating between an external surface of said housing and an internal surface of said housing.
